(19) United States
(12) Patent Application Publication
(10) Pub. No.: US 2009/0018580 A1
(43) Pub. Date: Jan. 15, 2009

(54) Surgical Method and Apparatus

(76) Inventor: Allan E. Wulc, Bryn Mawr, PA (US)

Correspondence Address: John F. McNulty;Paul & Paul, 2900 Two Thousand Market Street, Philadelphia, PA, 19103, US

(21) Appl. No.: 12/170,517

(22) Filed: Jul. 10, 2008

Related U.S. Application Data
(60) Provisional application No. 61/036,496, filed on Mar. 14, 2008.
(60) Provisional application No. 60/949,625, filed on Jul. 13, 2007.

(51) Int. Cl. A61B 17/04; A61B 17/06

(52) U.S. Cl. 606/228; 606/223

(57) ABSTRACT

A surgical method and apparatus is provided for reducing the effects of aging skin. SSTFM under the skin is engaged through an incision by a suture needle that carries a suture. The sutured SSTFM is lifted, and sutured to a fixed facial structure such as a ligament, bone, periosteum or deep facia (hereby reattaching the tissue under the skin to the fixed facial structure.

### CROSS-REFERENCE TO RELATED APPLICATIONS

This application claims priority from provisional application No. 61/036,496 filed Mar. 14, 2008 and from provisional application No. 60/949,625 filed Jul. 13, 2007, the complete disclosures of which are hereby incorporated by reference.

### THE PRESENT INVENTION

In accordance with this invention, it has been found that aging occurs due to the loss of attachments of skin to the deeper subcutaneous tissues in the face and neck, especially the ligaments.

These ligaments, which are thick attachments arising from the bone and periosteum, include:
the zygomatic ligament in the cheek (loss of attachment of which explains sagging of the cheek fat pad)
the platysma auricular ligament (loss of attachment of which explains sagging of tissues in the neck)
the ligaments of the hyoid (loss of attachment of which explains sagging of tissues in the neck)
the superior temporal crest and inferior temporal crest ligaments in the brow and the temple (loss of attachment of which explains drooping of the brow)
the suspensory ligaments of the eyelid (Whitnall's ligament in the upper eyelid) (loss of attachment of which explains drooping of the upper eyelid)
the suspensory ligaments of the lower eyelid (Lockwood's ligament in the lower eyelid) (loss of attachment of which explains lower eyelid entropion and ectropion)
the orbicularis retaining ligament (loss of attachment of which explains sagging of the tissues under the eyelids)
the orbital malar ligament (loss of attachment of which explains lower eyelid festoons, bags on bags in cheek)
the mandibular ligaments (loss of attachment of which explains formation of the jowl).

In accordance with this invention, the correction of facial aging therefore does not involve removing skin as in a facelift, or tightening the muscles, as in what is called a SMAS facelift, but instead involves reattaching the subcutaneous soft tissue, fascia and muscle basically, the tissue underneath the skin, hereinafter referred to as SSTFM to the ligaments of the face, through a slit-like opening or incision in the skin, preferably above the hairline, from which the SSTFM has lost the previous tight attachment it had in youth.

Once the skin finds its reattachments through a surgical repair, the face should look as it did in youth, with the exception of the changes that occur in the skin as a result of sun damage (wrinkles, skin pigment, changes and the exception of excess fat deposits that occur with weight gain and changes in body fat percentage associated with aging).

U.S. Pat. No. 7,060,079, the complete disclosure of which is herein incorporation by reference, relates to a device that sutures subcutaneous soft tissue using a vacuum as a means of engaging the skin and a rubber sealing device as a means of allowing penetration of a needle through the vacuum to hold the vacuum in place.

The instrument disclosed in U.S. Pat. No. 7,060,079 can be used to engage the ligaments of the face, in other words, the deeper tissues, to secure the subcutaneous soft tissue fat and muscle, by turning the instrument in reverse direction and engaging the deeper ligamentous or SMAS structures and securing them to the SSTFM which has likewise been engaged with the instrument. When the suture that has been used to secure the subcutaneous fat, fascia and muscle is tied and secured in a knot to the suture that has been used to engage the ligament, a lift of the face is effected without the necessity of wide resection of skin. This allows for precise elevation and is an anatomic way of reestablishing skin-ligamenous reattachments. It is a new way of reversing the gravitational changes associated with facial aging. While a very skilled surgeon (an artisan) can accomplish this without such an instrument, this instrument enables a face lift to occur where such specialized skill may not be present.

### SUMMARY OF INVENTION

The present invention therefore involves a method of and device for reducing the effects of aging skin, by returning skin and its associated SSTFM (which includes SMAS, fat, fascia, muscle and connective tissue) to its attachment to deeper subcutaneous tissues in the face, in particular, the ligaments.

### BRIEF DESCRIPTIONS OF THE DRAWING FIGURES

FIG. 1 is a representative view of a face of a patient, with a slit incision shown therein, through which a lifting device is to be inserted.

FIG. 2 is a greatly enlarged sectional view of the lifting device shown inserted through the slit incision in the skin of FIG. 1, to a deeper distal portion under the skin and the SSTFM of the patient's face in which a vacuum can be drawn to pull a portion of the SSTFM that is under the skin of the patient's face into a sleeve-like arrangement of the device, whereby a needle can be inserted through the skin, through an elastomeric portion of the device, through the SSTFM, through another elastomeric portion of the device, and outwardly again through the SSTFM and skin, with the needle carrying a suture therewith, for suturing the device inside the SSTFM that is located under the skin of the patient's face.

FIG. 3 is a fragmentary illustration of an alternative placement of a curved needle that carries a suture, through the elastomeric sleeve and SSTFM, from right to left as viewed in FIG. 3.

FIG. 4 is an illustration similar to that of FIG. 2, but wherein the needle has performed its function and has been cut away, and the suture is now in place, connecting the SSTFM that is under the skin of the face of the patient, to the device inserted under that skin.

FIG. 5 is an illustration similar to that of FIG. 2, but wherein, as an alternative the needle passes through holes in a cup-like portion of the vacuum device in addition to passing through the elastomeric portion of the device.

FIG. 6 is an enlarged fragmentary portion of the skin and SSTFM of a patient, wherein the device that was inserted inside the outer skin and SSTFM has engaged the SSTFM with a suture, and wherein the needle that carried the suture has already been cut away, leaving the suture engaged as shown.

FIG. 7 is an illustration similar to that of FIG. 6, but wherein the ends of the suture have been pulled through the skin and SSTFM from their disposition as shown in FIG. 6, leaving the suture connected to the SSTFM as shown, with the ends of the suture still engaged with the elastomeric sleeve.

FIG. 8 is a fragmentary illustration of the ligament illustrated in FIGS. 2, 4 and 6, and wherein a suture has been engaged with that ligament, and wherein the SSTFM that has been engaged with a suture as shown in FIGS. 4 and 6, has been lifted upwardly, with the two sutures in proximity with each other, and wherein there is fragmentally illustrated, a suture connector device for engaging the suture at the right of FIG. 8, for that suture to engage the ligament.

FIG. 9 is an illustration similar to that of FIG. 8 (without the illustration of the suture connector device of FIG. 8), and wherein the two sutures are illustrated tied together, so that the face lift has been effected, whereby the SSTFM is reattached to the facial ligament.

FIG. 10 is a fragmentary illustration of a needle and a suture that is inserted through the SSTFM of a patient, from the inside, through an incision, for lifting the SSTFM of a patient upwardly for subsequent attachment to a ligament, similar to the manner illustrated for attaching to a ligament, in FIG. 6. In FIG. 10, a vacuum can draw the SSTFM into a cup, optionally, or the SSTFM of the patient can be pressed inwardly by means of the surgeon's finger or thumb, or by means of an external instrument, for the threaded needle to engage the SSTFM without a vacuum, if said option is desirable.

FIG. 11 is an illustration of the connection of a suture to a ligament, similar to the right side of FIG. 8, but wherein a fragmentally illustrated device as shown in FIG. 10, is turned about 180° from that shown in FIG. 10, and is used to vacuum engage and apply a suture to the ligament.

FIG. 12 is a fragmentary illustration of an alternative embodiment of the lifting device of this invention, and a fragmentary portion of the outer skin and associated SSTFM of a patient, in which the device is adapted to engage the SSTFM of a patient and lift the same upwardly, without requiring the drawing of a vacuum against the SSTFM of a patient.

FIG. 13 is a fragmentary illustration taken generally along the line XIII-XIII of FIG. 12, of an arcuate needle with attached suture, inserted through the external skin surface of a patient, through the associated SSTFM, through a pair of rubber-like or elastomeric members that are carried by a bar inserted inside a slit incision of the skin of a patient as shown in FIG. 1 for example, engaging SSTFM of a patient that is between a pair of elastomeric members carried by the bar or rod, with the needle then passing outwardly through the SSTFM of a patient and its associated skin, wherein the suture remains in place between the captured portion of the SSTFM and the elastomeric members on each side of the bar, for lifting the SSTFM of a patient and the associated skin upwardly, by lifting the bar or rod upwardly, for subsequent attachment to a ligament in the manner illustrated in FIGS. 8 and 9.

### DETAILED DESCRIPTIONS OF THE PREFERRED EMBODIMENTS

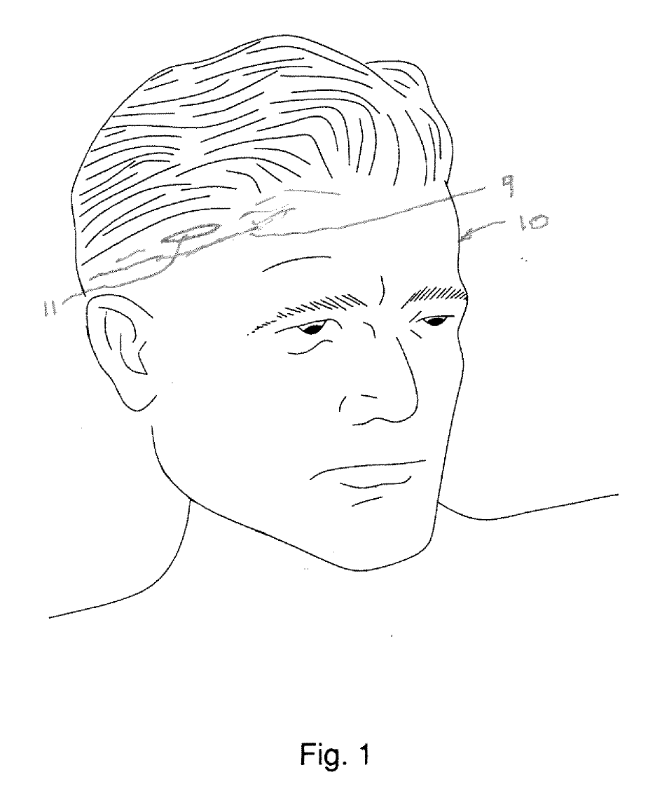

Referring to FIG. 1 in detail, it will be seen that there is the face of a patient generally designated by the numeral 10, having a slit incision 11 that has been cut through the external skin of the face of the patient, generally along a portion of the temple of the patient, above the hairline 9.

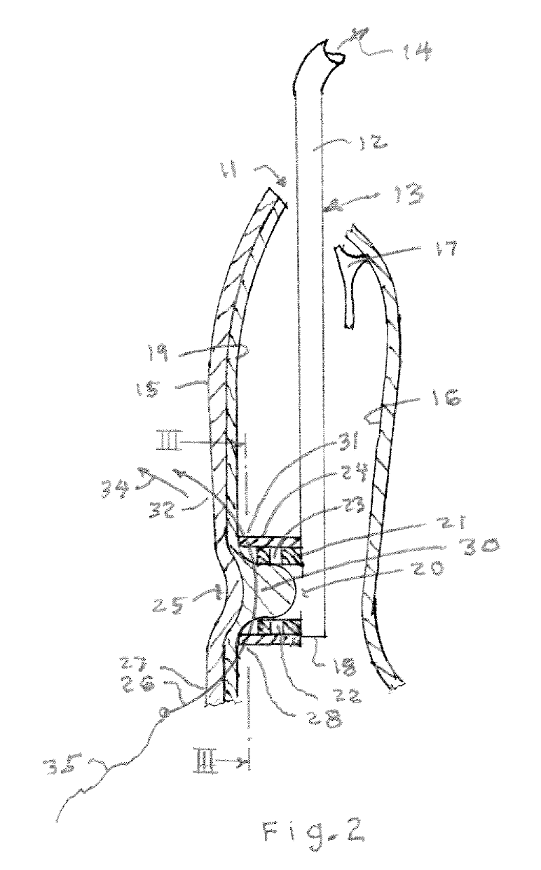

With reference now to FIG. 2, it will be seen that the slit incision 11 has had a suction tube 12 inserted therein, as part of the lifting device generally designated by the numeral 13, for drawing a vacuum thereon in the direction of the arrow 14, by any appropriate means, such as by line vacuum as normally is present in a hospital, surgicenter or physician's office, or any other vacuum device.

The tube 12 is inserted inside the SSTFM 19 and under the skin 15 of the face of a patient, and outside other subcutaneous tissue such as that 16. Inside the subcutaneous tissue is a representative ligament 17 naturally attached thereto. The tube 12 extends downwardly therefore between the SSTFM 19 and tissue 16, generally as shown in FIG. 2. The lower end 18 of the tube has an opening 20, communicating with a generally cylindrical sleeve 21 carried by the lower end 18 of the tube. The cylindrical sleeve 21 opens leftwardly, as shown in FIG. 2, and will preferably be provided with a pair of through-holes 22, 23. Disposed about the cylindrical sleeve 21, is an elastomeric sleeve 24, of a rubber, elastic, silicone, Tygon (trademark of Saint-Gobain) or other non-porous rubber-like type. By inserting the lower end 18 of the tube 12 inside the SSTFM 19 of a patient, as shown, and drawing a vacuum thereon from the upper end, while the lower end 18 of the device is pushed leftwardly as shown in FIG. 2, so that the left-most portion of the elastomeric sleeve 24 engages against the inner surface of the SSTFM 19, as shown, a vacuum is drawn against that portion of the SSTFM 19, which draws the SSTFM 19 into the cylindrical opening at the left end the elastomeric sleeve 24 and preferably also into the left end of the cylindrical sleeve 21, due to the suction provided by the vacuum at that zone of the SSTFM, so that the skin, as observed from the outside (the left side as illustrated in FIG. 2), can form, but does not always form, a depression 25 due to such suction.

The surgeon then knows, by observation of the depression 25, or by pulling the device 13 outside the incision 11 in the vertical direction, thus elevating the SSTFM 19, where to engage the inside of the SSTFM 19 and the elastomeric sleeve 24 with a suture. A preferably curved needle 26 is then inserted through the skin at a location 27, through the SSTFM 19 and elastomeric sleeve 24 at a location 28, through the inside of the SSTFM 19, at a location 30, and then through the elastomeric sleeve at a location 31, then outwardly through the SSTFM 19 and skin 15 at a location 32, with the point of the needle 26 passing therethrough in the direction of the arrow 34, with the needle 26 carrying a suture 35 therewith. Further pulling of the needle 26 outwardly in the direction of the arrow 34, will pull the needle completely through the lower end of the device 13 and outwardly of the skin, pulling the suture 35 therewith, capturing the SSTFM in the elastomeric sleeve 24, which closes against the suture 26 for lifting the SSTFM via the sleeve 24 and suture 26 when the device 13 is lifted.

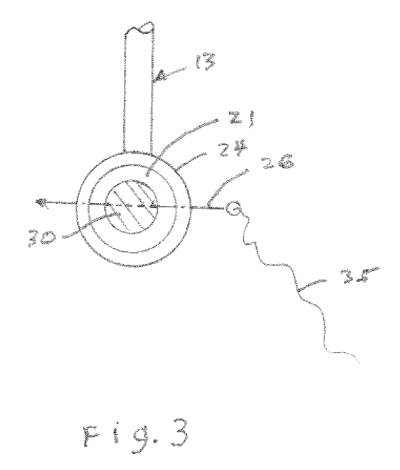

With reference to FIG. 3, taken generally along the line III-III of FIG. 2, it will be seen that there is provided an alternative orientation of placement of the arcuate needle 26 through the elastomeric sleeve 24, whereby the arcuate needle has entered the elastomeric sleeve from the right, carrying the suture 35 therewith, with the needle then passing through the SSTFM at its location 30, and then again passing through the elastomeric sleeve 24 at the left side thereof, such that the needle can be used to pull the suture 35 likewise through the elastomeric sleeve and captured SSTFM. This represents an alternative whereby the needle passes through the elastomeric sleeve essentially horizontally, rather than vertically as illustrated in FIG. 2.

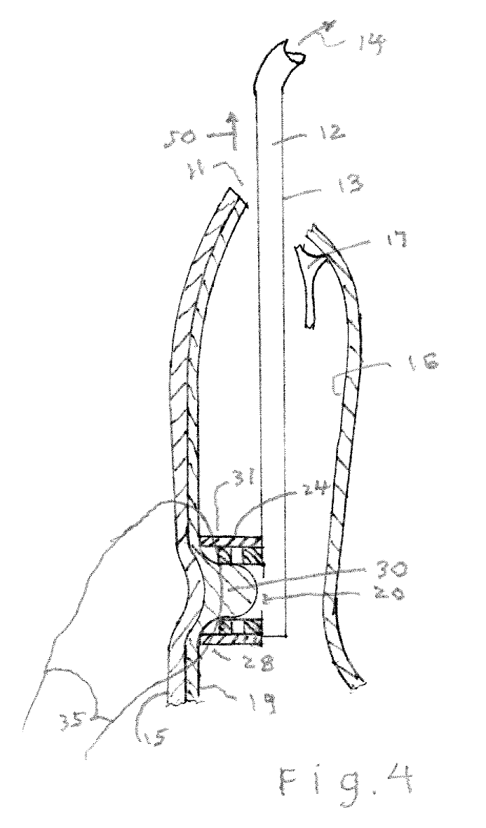

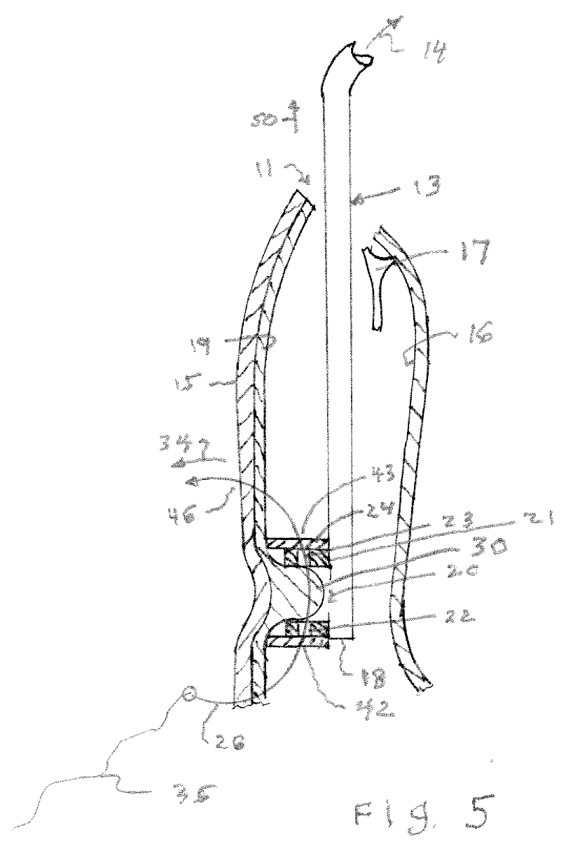

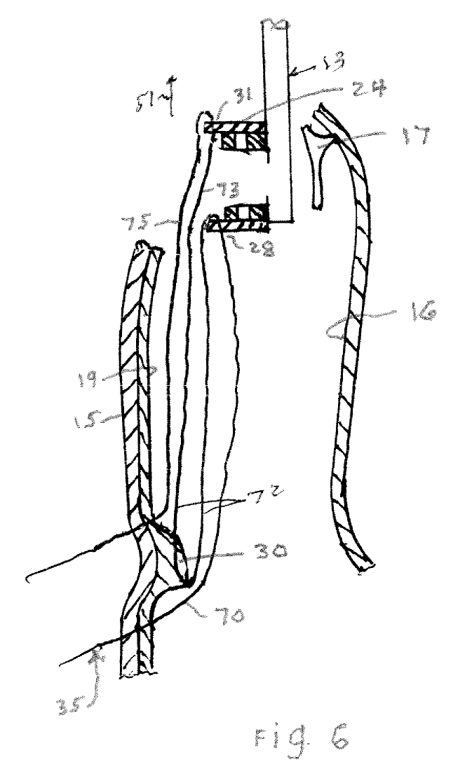

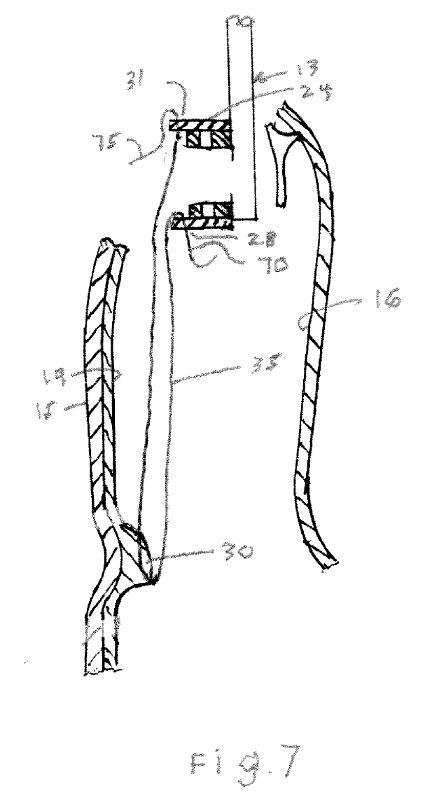

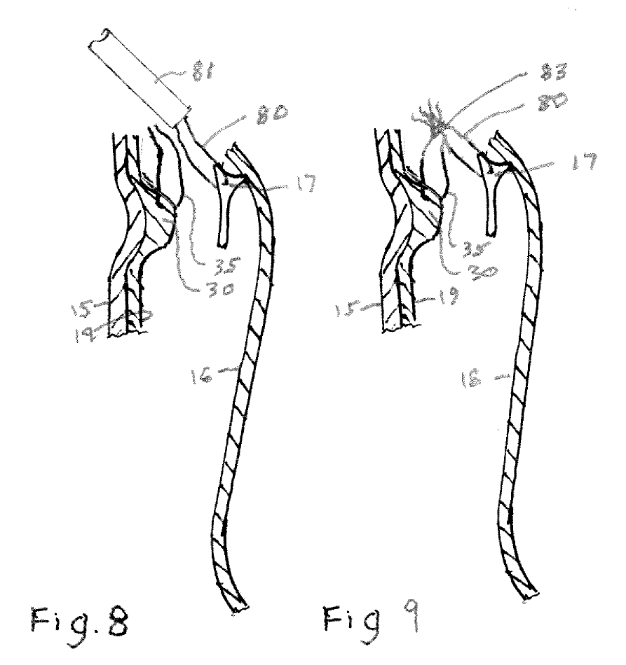

With reference now to FIG. 4, it will be assumed that a needle 26 (not shown) has been used in the process described above with respect to FIG. 2.

In FIG. 4, the suture 35 is shown, passing from outside the skin 15 through the SSTFM 19 under the skin 15, passing through the elastomeric sleeve 24, at the two locations 28, 31 capturing the SSTFM 19 at location 30, and then outwardly through the SSTFM 19 and skin 15 again. It will be noted that in FIG. 4, no arcuate needle is illustrated, in that, between the two steps illustrated in FIGS. 2 and 4, it is necessary that the arcuate needle has been cut away, leaving the suture 35, as illustrated, to enable withdrawal of the device out through the incision 11.

With reference to FIG. 5, the needle 26 can pass alternatively through the through-holes 22 and 23 of the cylindrical sleeve 21, as well as through the elastomeric sleeve 24, carrying a suture 35 therewith, with the curved needle 26 passing through the elastomeric sleeve 24 at locations 42, 43 and through the SSTFM 19 and out through the skin 15 at a location 46, to then be pulled through as was described above with respect to the needle 26 in FIG. 2, in the direction of the arrow 34.

Prior to lifting the device 13 upwardly, the vacuum being drawn at location 14 is discontinued and the device 13 is lifted upwardly through the slit incision 11 described above in the skin of the patient, in the direction of the arrow 50, with the elastomeric sleeve 24 carried by the device 13, gripping the suture 35 at two locations 28, 31 or 42, 43, and pulling the suture 35 upwardly with the device 13, and with the SSTFM and skin being threaded and engaged by the suture, as illustrated.

Referring now to FIG. 6, it will be seen that after the device 13 is lifted upwardly from its position of FIG. 4 in the direction of arrow 51, the suture 35 has one portion 70 thereof passing through the outer skin 15 and SSTFM 19 as shown, and is engaged with the elastomeric sleeve 24 at location 28, with another portion 72 of the suture then passing back through the SSTFM at a location 30, and then, with yet another portion 73 of the suture passing upwardly through the elastomeric sleeve 24 at another location 31 thereof, and with yet another portion 75 of the suture 35 then passing downwardly through the SSTFM 19 and skin 15, as shown.

With specific reference to FIG. 7, it will now be seen that the device 13 has been lifted upwardly, such that the legs 70 and 75 of the suture 35 have been drawn through the SSTFM 19 and skin 15, such that the SSTFM at location 30 is engaged and the suture is still engaged with the elastomeric sleeve 24 at the two locations 28 and 31 as shown.

With reference to FIG. 8 it will be seen that the suture 35 has been lifted upwardly, carrying the SSTFM at location 30 therewith. Such upward lifting can be effected manually, or with a lifting device 81, such as a suction device constructed similarly to the device 13, or as shown for example, in the device of U.S. Pat. No. 7,060,079.

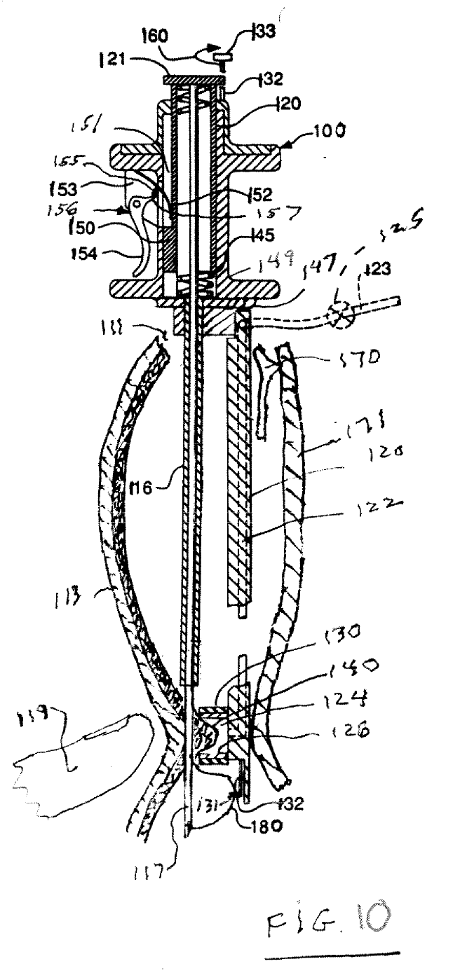

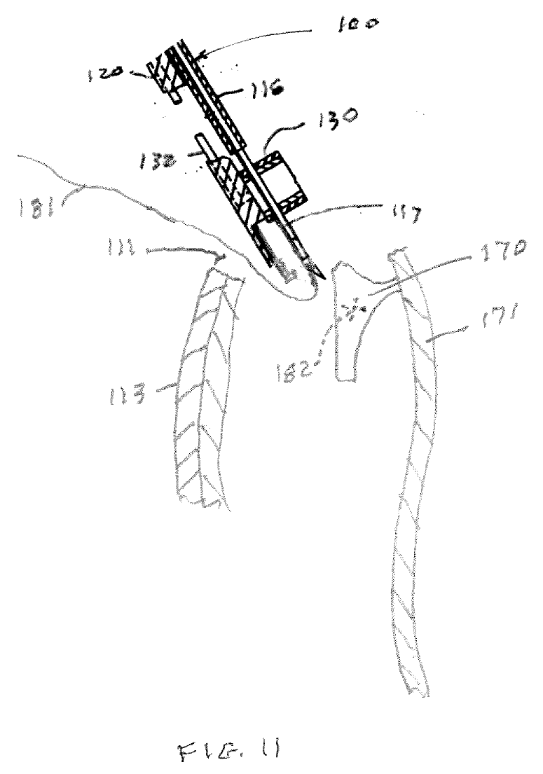

With further reference to FIG. 8, it will be seen that another suture 80 has engaged the ligament 17, as shown, such that the two sutures 35 and 80 are proximately adjacent each other. The means by which the suture 80 has engaged the ligament 17 can be an endoscope device 81 which has been inserted through the slit incision 11, visualizing the artisanal passage of the suture 80 therewith, and which engages the suture 80 with the ligament 17, if desired by means of a conventional surgical needle driver. Such an endoscope can be one of conventional construction as is known to cosmetic surgeons generally, which endoscopes usually carry a lens for viewing in addition to a source of light which can be connected to a camera and allow for either direct or video screen viewing. Alternatively, the means for suturing the suture 80 to the ligament 17 can be through the use of an instrument as shown in FIG. 10 hereof and as is disclosed in U.S. Pat. No. 7,060,079, but turned approximately 180° from the illustration of FIG. 10, as is addressed with reference to FIG. 11 hereof.

With reference now to FIG. 9, it will be seen that the sutures 35 and 80 are tied together, as is schematically shown at 83, and that the SSTFM that is engaged by the suture 35 at location 30, has now been lifted as shown and has now been re-attached to the ligament 17, achieving the previous tight attachment it had in youth.

Referring now to FIG. 10 in detail, it will first be understood that the device 100 will, in many respects, be constructed in a manner similar to, and having a structure similar to, that of U.S. Pat. No. 7,060,079, the complete disclosure of which is herein incorporated by reference.

The device or instrument 100 has a portion of its upper end in the shape of a spool 149, which remains outside the incision of access opening 111 of the patient, during use. Portions of the insert 120 near the lower end are shown broken away for the sake of clarity. The insert 120 includes a hollow sleeve 116 extending downwardly from the lower end of the spool 149. A suturing needle 117 is slideably carried in the sleeve 116, for upward and downward movement therein, with the needle 117 carrying a suture 180 at its lower end, which suture 180 enters through the access opening 111. The upper end of the needle 117 is carried by a vertically moveable plunger 120, being mounted to the upper end 121 of the plunger, for movement therewith, as the plunger 120 is engaged by the surgeon for downward movement thereof from the position of the plunger 120 prior to it being depressed by the surgeon engaging against the end 121, to move the needle into the position shown in FIG. 10.

The insert 120 optionally carries a vacuum conduit 122 therethrough, extending downwardly from a vacuum line 123 to a leftwardly opening cavity or cup 124. A manually actuable valve 125 can be provided for the vacuum line 123, for off/on actuation by the surgeon. The cavity 124 is comprised of cylindrical sleeve 126, and will preferably have a rubber, neoprene, elastomeric or similar sealing cylindrical sleeve 130 applied thereover.

When vacuum is used as shown in FIG. 10, it draws SSTFM 140 into the cup 124, and the needle 117 may pass through the SSTFM, as shown in FIG. 10, through the sleeve 126 that comprises the cavity 124.

A hook 131 is shown at the lower end of the insert 120, carried by a vertically disposed rod 132, which rod 132 extends from the upper end of the insert 120, up through the insert (not shown), passing through the instrument 100, to emerge at the upper end thereof and terminating in an actuation knob 133. After the needle 117 engages the SSTFM 190, the compression spring 145 urges the insert 120 upwards a slight amount, until the block 150 slides in keyway 151 until the block 150 engages the lower end of the leaf spring 152, allowing the suture 180 to form a loop, as shown, for ease of engagement of the hook 131 therewith. The actuation knob 133 may then be rotated as shown by the arrow 160 at the upper end of FIG. 10, to enable the hook 131 to engage the suture 180, such that when the sleeve 116 is lifted upwardly, the suture 180, thus engaged, will be able to lift the SSTFM 140 upwardly for attachment to a ligament 170, as will be described hereinafter with reference to FIG. 11.

With reference to FIG. 10, a helical compression spring 145 is shown, disposed about the upper end of the needle 117, inside the spool 149, in seated engagement against the lower end 147 of the spool 149, and pressing against a surface, in the same manner as comparable components are shown in U.S. Pat. No. 7,060,079, to urge the plunger upwardly. When the plunger 120 is engaged by the surgeon and pressed downwardly, the needle 117 likewise moves downwardly to engage the SSTFM as shown in FIG. 10. When the plunger moves downwardly, portion 150 slides in keyway 151. Also, when the plunger moves downwardly, it will engage the leaf spring 152. When the leaf spring 152 is moved leftwardly, by a finger of the surgeon engaging against surface 154 of the cam member 156, so that protrusion 155 carried by the leaf spring 152 is engaged by cam foot 157 of cam member 156, the leaf spring 152, being moved out of the way, enables the protrusion 150 to slide upwardly in the keyway, urged there by the compression spring 145. Alternatively to the use of a vacuum as described above, a thumb or finger 119 of the surgeon, or a plunger as shown in U.S. Pat. No. 7,060,079 may be used to create an indentation in the outer skin 113 of the patient, likewise for insertion of the needle 117 as shown in FIG. 10.

With the suture 180 thus engaged with the SSTFM 140, either with or without a vacuum applied, the SSTFM 140 may be lifted upwardly as the insert 120 is lifted upwardly, likewise lifting the suture 180 and SSTFM 140 as the device 100 is withdrawn through the incision 111.

Then, as shown in FIG. 11, the device 100 is rotated approximately 180° and is reinserted through the incision 111 and the needle 117 and the hook 131 may be used to secure a different suture 181 to the ligament 170 carried by the subcutaneous tissue 171, for example, through the spot 182 marked in phantom in FIG. 11 in the same manner that the suture 180 is secured to the SSTFM 140 in FIG. 10. Then the two sutures 180 and 181 can be tied together as shown for example in FIG. 9 for the sutures 35 and 80, to secure the upwardly lifted SSTFM to the ligament 170. Alternatively, the same suture 180 may be applied to the ligament 170.

It will also be apparent that the device of FIG. 10 can be used for non-endoscopic purposes; namely as a stand-alone instrument.

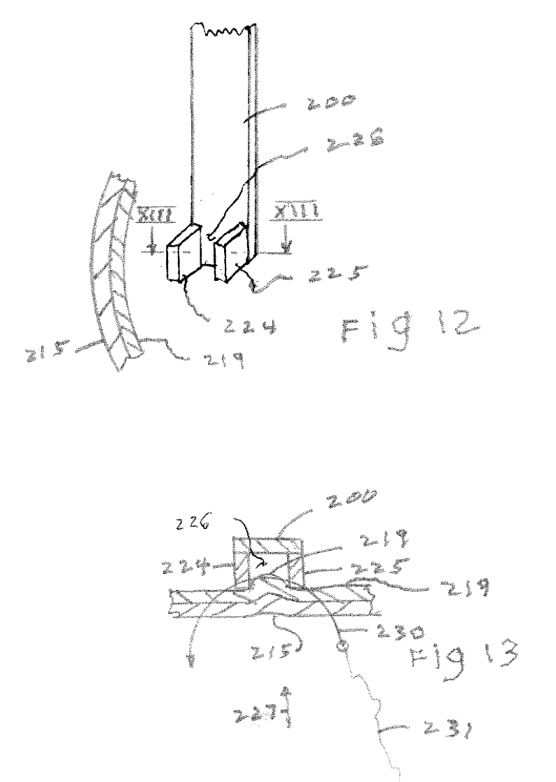

While engaging the SSTFM via a needle-carried suture as described above, with the SSTFM captured in the outer end of an elastomeric sleeve by drawing a vacuum on a device as described above is one way of practicing the present invention, it will be understood that the present invention can also be practiced the use of drawing of a vacuum on a device for engaging the SSTFM of a patient and lifting the skin associated therewith upwardly, as is addressed in FIGS. 12 and 13 hereof.

With reference now to FIG. 12, in the practiced hands of an experienced cosmetic surgeon, for example, a rod or bar 200 may be inserted through a slit incision 11 in the skin of a patient as disclosed in FIG. 1 carrying a pair of elastomeric members 224, 225 at the lower end thereof, forming a pocket 226 therebetween. The experienced surgeon may then press against the skin 215 of the patient in the direction and zone indicated by the arrow 227, until the SSTFM 219 of the patient enters the zone 226 between the elastomeric or rubber-like members 224, 225 carried by the bar or rod 200. Then, as shown in the illustration of FIG. 13, with the SSTFM 219 captured between the elastomeric members 224, 225 an arcuate needle 230 carrying a suture 231 may be drawn through the elastomeric members 224, 225 and captured SSTFM 219 as shown in FIG. 13, with the needle 230 pulling the suture 231 therethrough, until the needle exits the SSTFM 219 and skin 215, leaving the suture in engagement with the elastomeric members 224, 225 with the captured SSTFM 219 therebetween, to be followed by a lifting of the bar or rod 200 upwardly, for then suturing the captured SSTFM to a ligament by means of another needle and the suture carried therewith, in the same manner as illustrated and described with reference to FIGS. 8, 9 and/or 11.

In accordance with this invention, it will thus be seen that in effecting a face lift as described herein that differs from a conventional face lift, in that it does not require removal of, or cutting away skin material, but rather effects a lifting through a small slit such as that 11 of FIG. 1, by means of engaging SSTFM at a lower location in the face by means of an inserted member carrying elements therewith that are adapted to be engaged by a needle and a suture carried therewith, for attaching SSTFM at a lower location and lifting the same upwardly by lifting the inserted element upwardly, and then suturing the lifted SSTFM to a ligament, a face lift can be effected by a technique other than the conventional face lift technique that requires cutting away of skin.

The device for attaching the SSTFM to the inserted member that is to be lifted is preferably accomplished by capturing the SSTFM that is to be lifted between two members or portions of a single member that are elastomeric or rubber-like, such that a suture thus captured, with SSTFM between the elastomeric members, will remain engaged with the elastomeric member or portions of a single member while the instrument is drawn out of the face such that the sutures will be pulled through the external surface of the skin and remain engaged to the SSTFM while the instrument is drawn out, thus allowing a lifting to be effected, to allow a suture to be passed through the SSTFM. The instrument of U.S. Pat. No. 7,060,079 can be used, or, as described herein, a suture can be passed externally though two elastomeric members. The instrument of U.S. Pat. No. 7,060,079 or other instruments as disclosed herein may then be employed for a second pass with a second suture through a ligament. After withdrawing the instrument from the face following engagement of the ligament, the two sutures can be secured together allowing for reestablishing of the anatomy of youth by tying the lifted SSTFM to the ligament as described above.

What is claimed is:

1. A process of reducing or eliminating the effect of skin ptosis or drooping due to aging of a patient, by inserting a device under the outer skin and its adjacent SSTFM through a remote incision site, in order to engage the SSTFM, passing a suture through the SSTFM using the device, lifting the device and engaged SSTFM upwardly and removing the device from the patient, whereby, the suture thus passed through the SSTFM allows the SSTFM to be lifted upwardly, and then suturing the lifted SSTFM to a fixed facial structure such as a ligament, bone, periosteum, or deep facia.

2. The process of claim 1, wherein when the device is inserted inside the SSTFM and outer skin, a vacuum is drawn on it, pulling the SSTFM into the device, creating a recess or dimple that identifies to the surgeon the general location where the SSTFM has been drawn into the device, and inserting the suture at that location to suture the engaged SSTFM to the device.

3. The process of claim 2, wherein the device includes at least one suture-engaging sealing member, through which the vacuum is drawn, which suture-engaging sealing member engages the inside surface of the SSTFM under the skin, in vacuum-drawn tight engagement therewith.

4. The process of claim 1, wherein the device includes at least one suture-engaging sealing member and engaging the suture at two locations with the SSTFM that is to be lifted, between those locations.

5. The process of claim 1, including the step of rotating the device away from the surface of the skin and passing an additional suture through a fixed facial structure such as a ligament, including the further step of tying together the suture that passed through the SSTFM and the suture that is passed through the fixed facial structure, thereby reattaching the SSTFM to the fixed facial structure.

6. The process of claim 5, wherein the rotation of the device is through an arc of approximately 180°.

7. The process of claim 4, wherein the suture is passed through the skin of a patient and is captured in the at least one suture-engaging sealing member prior to lifting the device upwardly.

8. The process of claim 2, wherein the suture engages the at least one suture-engaging sealing member at two locations, with the SSTFM that is to be lifted being between those locations.

9. The process of claim 2, including the step of pulling on the device from a location external to the incision site to facilitate locating where the device engages the SSTFM.

10. The process of any one of claims 3, 4, 7 and 8, wherein the at least one suture-engaging sealing member is elastomeric.

11. The process of claim 4, wherein the suture is passed through the SSTFM via the remote incision site.

12. The process of claim 1, including the step of passing an additional suture through a fixed facial structure such as a ligament, including the further step of tying together or securing by other means, such as a knot pushing device, the suture that is passed through the SSTFM and the suture that is passed through the fixed facial structure, thereby reattaching the SSTFM to a fixed facial structure.

13. An apparatus for reducing or eliminating the effect of skin ptosis or drooping due to aging of a patient comprising a device for insertion under the outer skin and its adjacent SSTFM through a remote incision site, in order to engage the SSTFM, a suture needle and suture for passing through the SSTFM using the device for lifting the device and engaged SSTFM upwardly and means for then suturing the lifted SSTFM to a fixed facial structure such as a ligament.

14. The apparatus of claim 13, including means for drawing a vacuum on the device for pulling the SSTFM into the device and creating a recess or dimple that identifies to the surgeon the general location where the SSTFM has been drawn into the device, for inserting the suture at that location to suture the engaged SSTFM to the device.

15. The apparatus of claim 14, wherein the device includes at least one suture-engaging sealing means, through which the vacuum is drawn, whereby the suture-engaging sealing means engages the inside surface of the SSTFM under the skin, in vacuum-drawn tight engagement therewith.

16. The apparatus of claim 13, wherein the device includes at least one suture-engaging sealing means for engaging a suture at two locations with the SSTFM that is to be lifted, between those locations.

17. The apparatus of claim 13, including a second suture needle and suture for application through a fixed facial structure such as a ligament, including means for tying together or securing the two sutures together by other means such as a knot pushing device, the suture that passed through the SSTFM and the second suture that is passed through the fixed facial structure, thereby reattaching the SSTFM to the fixed facial structure.

18. The apparatus of any one of claims 15 and 16, wherein the at least one suture-engaging sealing means is elastomeric.

19. The apparatus of claim 13, for use for any of:
(a) endoscopic purposes:
(b) non-endoscopic purposes.
